(19) United States
(12) Patent Application Publication
(10) Pub. No.: US 2015/0282790 A1
(43) Pub. Date: Oct. 8, 2015

(54) ENDOSCOPIC CLOSURE DEVICE

(71) Applicant: Boston Scientific Scimed, Inc., Maple Grove, MN (US)

(72) Inventors: Daniel R. QUINN, Littleton, MA (US); Michael E. Zupkofska, Rockland, MA (US); Kenny J. King, Somerville, MA (US); Rachael A. Rheaume, Framingham, MA (US); Kerry L. Grant, Northbidge, MA (US); John A. Hingston, Framingham, MA (US); Brian Gaffney, Rutland, MA (US)

(21) Appl. No.: 14/678,523

(22) Filed: Apr. 3, 2015

Related U.S. Application Data
(60) Provisional application No. 61/976,861, filed on Apr. 8, 2014.

(51) Int. Cl. A61B 17/00

(52) U.S. Cl. CPC A61B 17/0057; A61B 17/00234; A61B 2017/00659; A61B 2017/00862

(57) ABSTRACT

A tissue closure device includes a clip including arms extending from proximal ends connected to one another to distal ends. The clip is biased toward an open configuration in which the distal ends extend away from a longitudinal axis of the clip. The arms are held in a closed configuration in which the arms are moved toward the longitudinal axis via an interior surface of a working channel through which the clip is passed until the clip is moved distally past a distal end of the working channel and permitted to revert to the biased open configuration so that the distal ends are positioned about a portion of tissue surrounding a tissue opening to be closed. The device also includes a locking element movable over the arms to lock the clip in the closed configuration in which the arms are drawn toward one another to grip tissue therebetween.

### PRIORITY CLAIM

The present disclosure claims priority to U.S. Provisional Patent Application Ser. No. 61/976,861 filed on Apr. 8, 2014; the disclosure of which is incorporated herewith by reference.

### BACKGROUND

Physicians have become increasingly willing to perform more aggressive interventional and therapeutic endoscopic procedures including, for example, removal of larger lesions (e.g., cancerous masses), tunneling under a mucosal layer of the gastro-intestional (GI) tract to treat tissues below the mucosa, full thickness removal of tissue, inserting devices through the GI tract and then penetrating the GI organ to treat tissue outside the GI tract, and endoscopic treatment/repair of post-surgical issues (e.g., post-surgical leaks, breakdown of surgical staple lines, anastomotic leaks). These procedures may increase the risk of perforating the wall of the GI tract, or may require closure of the GI tract wall as part of the procedure. Endoscopic closure reduces cost and may reduce patients' trauma, pain and inconvenience. However, current tissue closure devices may be insufficient to close certain perforations.

### SUMMARY

The present disclosure is directed to a tissue closure device. The tissue closure device comprises a clip including a plurality of arms extending from proximal ends connected to one another to distal ends, the clip being biased toward an open configuration in which the distal ends extend away from a longitudinal axis of the clip, the arms being held in a closed configuration in which the arms are moved toward the longitudinal axis via an interior surface of a working channel through which the clip is passed until the clip is moved distally past a distal end of the working channel and permitted to revert to the biased open configuration so that the distal ends are positioned about a portion of tissue surrounding a tissue opening to be closed and a locking element movable over the arms to lock the clip in the closed configuration in which distal ends of the arms are drawn toward one another to grip tissue therebetween.

### BRIEF DESCRIPTION

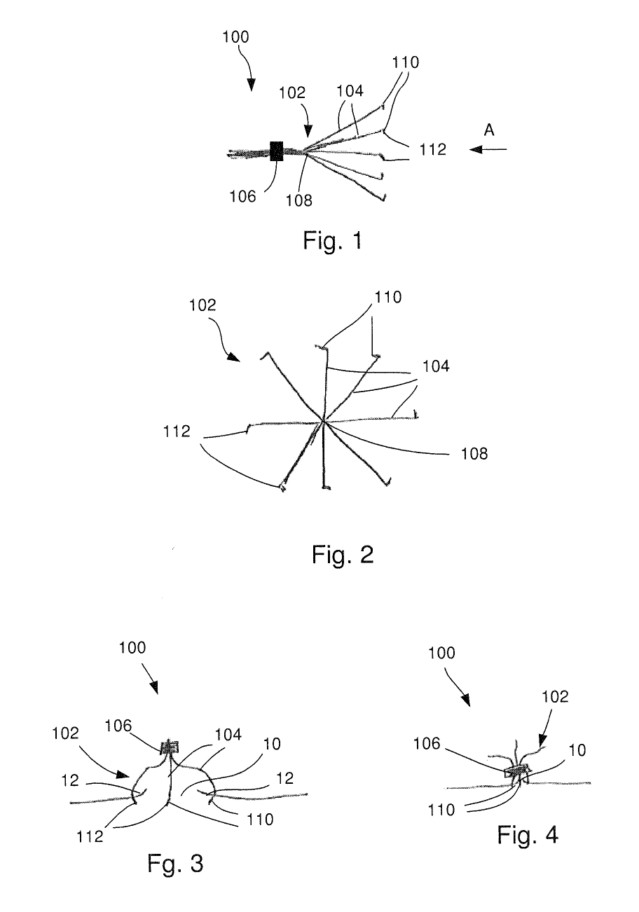

FIG. 1 shows a side view of a device according to a first exemplary embodiment of the present disclosure, in a first configuration;

FIG. 2 shows a plan view of the device of FIG. 1, in a direction A;

FIG. 3 shows a side view of the device of FIG. 1, in an unlocked configuration;

FIG. 4 shows a side view of the device of FIG. 1, in a locked configuration;

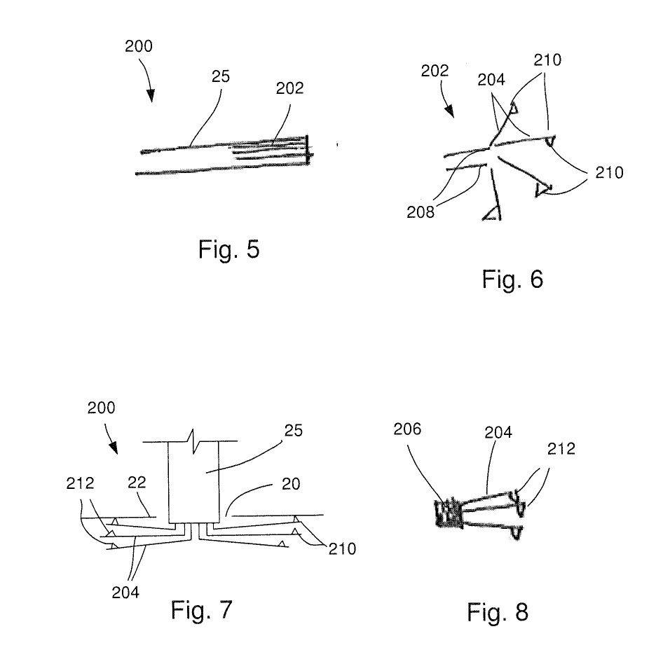

FIG. 5 shows a side view of a device according to a second exemplary embodiment, in an insertion configuration;

FIG. 6 shows a side view of the device of FIG. 5, in an open configuration;

FIG. 7 shows a side view of the device of FIG. 6, in the open configuration and engaging a target tissue;

FIG. 8 shows a side view of the device of FIG. 6, in a locked configuration; and

### DETAILED DESCRIPTION

The present disclosure may be further understood with reference to the following description and the appended drawings, wherein like elements are referred to with the same reference numerals. Exemplary embodiments of the present disclosure describe endoscopic devices. In particular, the exemplary embodiments describe endoscopic clipping devices for tissue closure. The tissue clipping devices of the present disclosure may be passed through a working channel of an endoscopic inserted into a GI tract and deployed from a distal end thereof to clip target tissue. Although exemplary embodiments of the present disclosure specifically describe a device for treating the GI tract, it will be understood by those of skill in the art that devices of the present disclosure may be used to clip tissue in any of a variety of types and locations of tissue. It should also be noted that the terms “proximal” and “distal” as used herein refer to a direction toward (proximal) and away from (distal) a user of the device.

As shown in FIGS. 1-4, a device 100 according to a first exemplary embodiment comprises a clip 102 including a plurality of arms 104 movable between an open configuration in which the arms 104 are spaced from one another to receive target tissue and a closed configuration in which the arms 104 are drawn together to grip the target tissue. A locking element 106 movably mounted over the clip 102, is operable to lock the arms 104 in the closed configuration. As would be understood by those skilled in the art, the device 100 is sized and shaped and sufficiently flexible to permit its insertion through a working channel of an endoscope along a tortuous path (e.g., though a body lumen accessed via a naturally occurring body orifice) to a target tissue. When the clip 102 is within the working channel, an interior surface of the working channel holds the arms 104 in the closed configuration so that the clip 102 may be inserted into the body in the closed configuration. The arms 104 are biased toward the open configuration so that, when a target site is reached, the clip 102 may be moved distally out of a distal end of the working channel permitting the arms 104 move to the open configuration. The clip 102 is then positioned so that the arms 104 surround a portion of tissue to be clipped (e.g., tissue surrounding a tissue opening 10 or a wound to be closed). When the arms 104 are in a desired position engaging the tissue to be clipped, the locking element 106 is slid distally over the arms 104 to draw the arms 104 into the closed configuration to grip the target tissue (e.g., to close the tissue opening 10). The locking element 106 may then be fixed over the arms 104 to lock the clip 102 in the closed configuration.

The clip 102 may be actuated and deployed in the body via, for example, a pusher or other actuating element moving the device 100 distally through the working channel until the clip 102 reaches the target site. The pusher may extend longitudinally from a proximal end connected to a handle portion accessible to a user, when the clip 102 is inserted into the body, to a distal end connected to the clip 102. For example, arms 104 of the clip 102 may be formed of a suture or other flexible material such as, a wire which is connected to a distal end of a pusher. The arms 102 of the clip 102 may, for example, be wound about a distal portion of the pusher. It will be understood by those of skill in the art, however, that the arms 104 may be connected to the pusher in any of a variety of ways. In another exemplary embodiment the arms 102 may be adhered to the pusher, clamped to the pusher or connected to the pusher via a frangible link designed to fail when a force thereon exceeds a predetermined threshold level. Once distal ends of the arms 104 have been positioned over the target tissue and locked in the closed configuration via the locking element 106, the clip 102 may be deployed in the body in the locked, closed configuration. For example, a cutting mechanism included therein may cut a proximal portion of the arms 104 connected to the pusher, to deploy the clip 102 in the body. In another embodiment, the user may draw the pusher proximally until the frangible link connecting the pusher and the clip 102 is broken. In yet another embodiment, the pusher may be rotated about a longitudinal axis thereof, via the handle portion, to disengage clip arms 104 wound about the distal end thereof. Although the device 100 is described as being inserted into the body via a working channel of an endoscope, it will be understood by those of skill in the art that the device 100 is not required to be inserted into the body via an endoscope. For example, the device 100, may be inserted into the body via an introducer tube or through the lumen of any other suitable device.

Each of the arms 104 extends from a proximal end 108 connected to the proximal ends 108 of the other arms 104 to a distal end 110. In the open configuration, the distal ends 110 of the arms 104 are separated from one another so that the distal ends 110 may be positioned about target tissue to be gripped (e.g., a tissue opening 10 to be closed). For example, the distal ends 110 may be positioned along edges 12 of the tissue opening 10. In the closed configuration, the distal ends 110 are drawn toward one another to draw edges 12 of the tissue opening 10 together, thereby closing the wound. The distal ends 110 may include a tissue engaging feature 112 enhancing the gripping engagement between the arms 104 and the tissue. The engaging features 112 may extend laterally inward relative to the arms 104 such that, when the arms 104 are in the closed configuration, each of the engaging features 112 extends toward the other arms 104. In one exemplary embodiment the engaging features 112 may be, for example, T-shaped elements transverse to a length of the arms 104 increasing an area over which the arms 104 engage gripped tissue. In another exemplary embodiment, the engaging features 112 may comprise tines extending radially inward from the distal end 110 to penetrate clipped tissue. It will be understood by those of skill in the art, however, that the arms 104 may include any of a variety of engaging features 112 so long as the arms 104 are capable of gripping and remaining coupled to target tissue to maintain the gripped tissue in a desired spatial relationship (e.g., to close the tissue opening 10 until natural healing processes have connected the edges of the opening 10). The arms 104 may be metal tines biased toward the open configuration. However, as would be understood by those skilled in the art, the arms 104 maybe formed of any suitable biocompatible and/or biodegradable materials.

The locking element 106 is slidably mounted over the clip 102 so that it may be longitudinally moved over the arms 104 from a proximal position in which the arms 104 are free to move to the open configuration and a distal position in which the arms 104 are constrained in the closed configuration. That is, once the clip 102 has been positioned over a target site such as the tissue opening 10, the locking element 106 is moved distally over the arms 104 to draw the arms 104 together into the closed configuration, closing the tissue opening 10. After the clip 102 has been moved to the closed configuration gripping tissue as desired, the locking element 106 is locked over the arms 104 to lock the clip 102 in the closed configuration. The locking element 106 may comprise, for example, a crimp which may be crushed over the arms 104, a locking ring locked over the arms 104 via a friction fit, a zip tie cinched over the arms 104 or a suture tied over the arms 104. It will be understood by those of skill in the art, however, that the locking element 106 may include any mechanism capable of maintaining the arms 104 over the gripped tissue in the closed configuration. In another embodiment, the clip 102 may be moved to the closed configuration by moving the clip 102 proximally relative to the working channel through which it has been inserted. Once the arms 104 of the clip 102 have been moved to the closed configuration, the locking element 106 may be moved over the arms 104 to lock the arms 104 in the closed configuration.

According to an exemplary surgical technique using the device 100, the device 100 is inserted to a target site within the body through the working channel of an endoscope inserted, for example, along a tortuous path through a body lumen accessed via a naturally occurring body orifice. The clip 102 is inserted into the body in the closed configuration, the arms 104 of the clip 102 held in the closed configuration via either the locking element 106 or through contact with an interior surface of the working channel. Upon reaching the target site, the arms 104 are moved distally out of the distal end of the working channel permitting the arms 104 to move to the open configuration under their natural bias. If the locking element 106 has been used to maintain the arms 104 in the closed configuration, the locking element 106 is moved proximally to free the arms 104 to move to the open configuration under their natural bias. As shown in FIG. 3, clip 102 is then positioned so that the distal ends 110 of the arms 104 are located about the tissue to be gripped (e.g., adjacent to tissue surrounding the opening 10). The clip 102 may then be advanced distally so that the engaging features 112 at the distal ends 110 of the arms 104 engage the target tissue. When the engaging features 112 have engaged the target tissue, the clip 102 is drawn into the closed configuration by moving the locking element 106 distally over the arms 104, as shown in FIG. 4. Closing the clip 102 draws the edges 12 of the tissue opening 10 together, thereby closing the tissue opening. When the desired tissue has been gripped between the arms 104, the locking element 106 is fixed over the arms 104 to lock the clip 102 in the closed configuration, holding the target tissue (e.g., closing the tissue opening 10).

Although the exemplary embodiment of the clip 102 has been shown and described as being closed via the locking element 106, it will be understood by those of skill in the art that the clip 102 may alternatively be closed by drawing the clip 102 proximally relative to the working channel. As the arms 104 are drawn into the working channel, the arms 104 will be drawn toward one another. Once the arms 104 have been drawn closed, the locking element 106 may be slid over the arms 104 to be locked thereover.

As shown in FIGS. 5-8, a device 200 according to a second exemplary embodiment of the present disclosure is substantially similar to the device 100 described above, comprising a clip 202 and a locking element 206 for locking the clip 202 in a closed configuration. The clip 202 is substantially similar to the clip 102, including arms 204 extending from a proximal end 208 connected to one another to a distal end 210. The clip 202 is biased toward an open configuration, as shown in FIG. 6, in which the arms 204 extend radially outward from a proximal end of the clip 202. Similarly to the clip 102, the distal end 210 of each of the arms 204 includes an engaging feature 212 (e.g., one or more tines, teeth, tags, etc.) for engaging the target tissue. In this embodiment, however, the engaging features 212, face proximally when the clip 202 is in the open configuration. The locking element 206 is also substantially similar to the locking element 106 and is mountable over the arms 204 to lock the clip 202 in the closed configuration. The locking element 206 may be, for example, a clip, cinch, locking ring, suture, etc.

An exemplary surgical technique of the device 200 is substantially similar to the surgical technique of the device 100. A distal end of the tube 25 of the device 200 is inserted through a tissue opening 20 to be closed so that the clip 202 is on a distal side of the tissue opening 20 in the closed configuration. When the tube 25 is positioned as desired, the clip 202 is moved distally out of the tube 25 permitting the arms 204 to move to the open configuration under their natural bias.

At this point, the clip 202 is drawn proximally to drive the engaging features 212 at the distal ends 210 into the target tissue (e.g., edges 22 of the tissue opening 20) so that the engaging features 212 penetrate and are locked into the target tissue. The clip 202 is then drawn proximally back into the tube 25 to draw the arms 204 together into the closed configuration drawing the edges 22 together to close the opening 20. Once the clip 202 has been moved into the closed configuration, the locking element 206 is slid distally over the arms 204 to lock the clip 202 in the closed configuration, as shown in FIG. 8, and the clip 202 is separated from the device 200. Similarly to the device 100, proximal ends of the arms 204 may be connected to a pusher and severed therefrom, in any of a number of ways, as described above in regard to the device 100. In one example, the clip 202 may be connected to the pusher via a frangible link that is designed to fail when a force exerted thereon exceeds a predetermined threshold level. In another example, the pusher may be rotated about a longitudinal axis thereof to unwind proximal ends of the clip arms 204 which are wound about a distal portion of the pusher. In yet another example, a cutting mechanism may be used to cut the arms 204 from the pusher to deploy the clip 202 in the body.

It will be apparent to those skilled in the art that various modifications and variations may be made in the structure and the methodology of the present disclosure, without departing from the spirit or scope of the disclosure. Thus, it is intended that the present disclosure cover modifications and variations of the disclosure provided that they come within the scope of the appended claims and their equivalents.

What is claimed is:

1. A tissue closure device, comprising:
a clip including a plurality of arms extending from proximal ends connected to one another to distal ends, the clip being biased toward an open configuration in which the distal ends extend away from a longitudinal axis of the clip, the arms being held in a closed configuration in which the arms are moved toward the longitudinal axis via an interior surface of a working channel through which the clip is passed until the clip is moved distally past a distal end of the working channel and permitted to revert to the biased open configuration so that the distal ends are positioned about a portion of tissue surrounding a tissue opening to be closed; and
a locking element movable over the arms to lock the clip in the closed configuration in which distal ends of the arms are drawn toward one another to grip tissue therebetween.

2. The device of claim 1, wherein the distal ends of the arms include an engaging feature for engaging the portion of tissue surrounding the tissue opening.

3. The device of claim 2, wherein the engaging feature includes one of teeth, tines and T-shaped elements.

4. The device of claim 2, wherein, when the clip is in the open configuration, the engaging feature extends toward the distal end of the device.

5. The device of claim 2, wherein, when the clip is in the open configuration, the engaging feature extends toward the proximal end of the device.

6. The device of claim 1, wherein the locking element is one of a locking ring, a crimp, a cinch and a suture.

7. The device of claim 1, wherein the arms include metallic sutures.

8. The device of claim 1, wherein, in the open configuration, the arms extend radially outward from a center of the device in an array configuration.

9. The device of claim 1, wherein the locking element is longitudinally slidable over the arms to move the clip from the open configuration to the closed configuration.

10. The device of claim 1, wherein the clip is sized and shaped to be inserted through a working channel of an endoscope.

11. A tissue closure system, comprising:
a clip including a plurality of arms extending from proximal ends connected to one another to distal ends, the clip being biased toward an open configuration in which the distal ends extend away from a longitudinal axis of the clip, the arms held in a closed configuration in which the arms are moved toward the longitudinal axis via an interior surface of a working channel through which the clip is passed until the clip is moved distally past a distal end of the working channel and permitted to revert to the biased open configuration so that the distal ends are positioned about a portion of tissue surrounding a tissue opening to be closed;
a pusher rod extending longitudinally from a proximal end connected to a handle portion accessible by a user, when the clip is inserted into the body, to a distal end connected to proximal ends of the arms; and
a locking element movable over the arms to lock the clip in the closed configuration in which distal ends of the arms are drawn toward one another to grip tissue therebetween.

12. The system of claim 11, wherein arms of the clip are flexible metallic sutures, proximal ends of which are wound about the distal end of the pusher.

13. The system of claim 11, wherein the clip is connected to the pusher via a frangible link.

14. The system of claim 11, further comprising a cutter for cutting a proximal portion of the arms to disengage the clip from the pusher.

15. The system of claim 11, wherein the distal ends of the arms include an engaging feature for engaging the portion of tissue surrounding the tissue opening.

16. A tissue closure device, comprising:
a clip including a plurality of arms extending from proximal ends connected to one another to distal ends, the clip being biased toward an open configuration in which the distal ends extend away from a longitudinal axis of the clip, the arms being held in a closed configuration in which the arms are moved toward the longitudinal axis via an interior surface of a working channel through which the clip is passed until the clip is moved distally past a distal end of the working channel and permitted to revert to the biased open configuration so that the distal ends are positioned about a portion of tissue surrounding a tissue opening to be closed; and
a locking element movable over the arms to lock the clip in the closed configuration in which distal ends of the arms are drawn toward one another to grip tissue therebetween.

17. The device of claim 16, wherein the distal ends of the arms include an engaging feature for engaging the portion of tissue surrounding the tissue opening.

18. The device of claim 17, wherein the engaging feature includes one of teeth, tines and T-shaped elements.

19. The device of claim 17, wherein, when the clip is in the open configuration, the engaging feature extends toward the distal end of the device.

20. The device of claim 17, wherein, when the clip is in the open configuration, the engaging feature extends toward the proximal end of the device.

21. The device of claim 16, wherein the locking element is one of a locking ring, a crimp, a cinch and a suture.

22. The device of claim 16, wherein the arms include metallic sutures.

23. The device of claim 16, wherein, in the open configuration, the arms extend radially outward from a center of the device in an array configuration.

24. The device of claim 16, wherein the locking element is longitudinally slidable over the arms to move the clip from the open configuration to the closed configuration.

25. The device of claim 16, wherein the clip is sized and shaped to be inserted through a working channel of an endoscope.

26. A tissue closure system, comprising:
a clip including a plurality of arms extending from proximal ends connected to one another to distal ends, the clip being biased toward an open configuration in which the distal ends extend away from a longitudinal axis of the clip, the arms being held in a closed configuration in which the arms are moved toward the longitudinal axis via an interior surface of a working channel through which the clip is passed until the clip is moved distally past a distal end of the working channel and permitted to revert to the biased open configuration so that the distal ends are positioned about a portion of tissue surrounding a tissue opening to be closed;
a pusher rod extending longitudinally from a proximal end connected to a handle portion accessible by a user, when the clip is inserted into the body, to a distal end connected to proximal ends of the arms; and
a locking element movable over the arms to lock the clip in the closed configuration in which distal ends of the arms are drawn toward one another to grip tissue therebetween.

27. The system of claim 26, wherein arms of the clip are flexible metallic sutures, proximal ends of which are wound about the distal end of the pusher.

28. The system of claim 26, wherein the clip is connected to the pusher via a frangible link.

29. The system of claim 26, further comprising a cutter for cutting a proximal portion of the arms to disengage the clip from the pusher.

30. The system of claim 26, wherein the distal ends of the arms include an engaging feature for engaging the portion of tissue surrounding the tissue opening.

31. A method for closing a tissue opening, comprising:
inserting a clip through a working channel of an endoscope to a target site within a body, the clip including a plurality of arms extending from proximal ends connected to one another to distal ends, the clip being biased toward an open configuration in which the distal ends extend away from a longitudinal axis of the clip, the arms being held in a closed configuration in which the arms are moved toward the longitudinal axis via an interior surface of the working channel until the clip is moved distally past a distal end of the working channel and permitted to revert to the biased open configuration;
positioning the distal ends of the arms about a portion of tissue surrounding a tissue opening;
moving the clip to the closed configuration such that the distal ends are moved toward one another to draw edges of the tissue opening toward one another;
moving a locking element over the plurality of arms to lock the clip in the closed configuration; and
deploying the clip in the body in the closed configuration.

32. The method of claim 31, wherein the arms are positioned about the tissue opening such that engaging features at distal ends of the arms engage tissue on a proximal side of the tissue opening.

33. The method of claim 31, wherein the arms are positioned about the tissue opening such that engaging features at distal ends of the arms engage tissue on a distal side of the tissue opening.

34. The method of claim 31, wherein inserting the clip includes moving a pusher longitudinally relative to the working channel, proximal ends of the arms connected to a distal end of the pusher.

35. The method of claim 31, wherein deploying the clip includes one of cutting the arms from the pusher and breaking a frangible link connecting the arms to the pusher.
